(19) United States
(12) Patent Application Publication
(10) Pub. No.: US 2010/0247588 A1
(43) Pub. Date: Sep. 30, 2010

(54) Method For Preparing Finely Divided Emulsions

(75) Inventors: Matthias Hloucha, Koln (DE); Esther Kusters, Duesseldorf (DE); Jasmin Menzer, Langenfeld (DE); Wolf Eisfeld, Langenfeld (DE)

Correspondence Address: FOX ROTHSCHILD LLP, 997 Lenox Drive, Bldg. #3, Lawrenceville, NJ, 08648, US

(73) Assignee: COGNIS IP MANAGEMENT GMBH, Duesseldorf (DE)

(21) Appl. No.: 12/665,494

(22) PCT Filed: Jun. 14, 2008
(86) PCT No.: PCT/EP2008/004802

§ 371 Date: Jun. 18, 2010

(30) Foreign Application Priority Data
Jun. 19, 2007 (EP) 07011967
Sep. 27, 2007 (DE) 10 2007 046575.2
Apr. 3, 2008 (DE) 10 2008 017032.1
Apr. 3, 2008 (DE) 10 2008 017034.8
May 7, 2008 (DE) 10 2008 022433.2

(51) Int. Cl. A61K 8/06; A61K 8/92; A61Q 5/02; A61Q 5/00; A61Q 15/00; A61Q 17/04

(52) U.S. Cl. 424/401

(57) ABSTRACT

Finely divided emulsions, preferably for use in cosmetic preparations can be produced by first producing a microemulsion which contains at least 10-20% by weight of an alkyl(oligo)glycoside of general formula R¹O-[G]ₚ wherein R¹ represents an alkyl group and/or alkenyl group with 4 to 22 carbon atoms, G represents a sugar with 5 or 6 carbon atoms and p is a number from 1 to 10, and 4-10% of an ester of glycerol with a fatty acid having a chain length of C12-C22 and 5-30% by weight of an oil body and the remainder to 100% by weight being constituted of water and optionally other ingredients, and then diluting said microemulsion with water amounting to 5 to 20 times the volume of the microemulsion at temperatures of 15 to 35° C.

The invention is in the field of cosmetic compositions which are present in the form of finely divided emulsions and further relates to a method for preparing such emulsions.

It is known that oil-in-water emulsions which are prepared with nonionic emulsifiers often suffer from phase inversion upon heating, i.e. at elevated temperatures, the external, aqueous phase can become the internal phase. This process is generally reversible, meaning that, upon cooling, the original emulsion type is reformed again. Emulsions which have been prepared above the phase inversion temperature generally have a low viscosity and high storage stability. WO 97/06870 A1 discloses sugar-surfactant-containing emulsions of this type. The preparation of such emulsions in practice, however, is complex and expensive since firstly an emulsion has to be heated and then cooled. Furthermore, there is a desire on the part of the consumer to directly produce finely divided emulsions, i.e. before or during application. Consequently, it is possible to dispense with additives which are customarily used for increasing the storage stability of finely divided emulsions.

The object of the present invention was therefore to provide aqueous finely divided emulsions which are easier to prepare.

The invention firstly provides the preparation of aqueous emulsions having an average particle size of less than 1 μm by firstly, in a first step, preparing a microemulsion comprising at least 10-20% by weight of an alkyl (oligo)glycoside of the general formula R¹O-[G]ₚ in which R¹ is an alkyl and/or alkenyl radical having 4 to 22 carbon atoms, G is a sugar radical having 5 or 6 carbon atoms and p is numbers from 1 to 10, and 4-10% by weight of an ester of glycerol with a fatty acid of chain length C12-C22 and 5-30% by weight of an oil body and as the remainder to 100% by weight, water and optionally further ingredients, and then, in a second step, diluting this microemulsion with water, using 5 to 20 times the volume of the microemulsion at temperatures of from 10 to 45° C., preferably from 15 to 35° C.

The method according to the invention is thus a two-stage process in which, in the first step, a microemulsion is prepared in a manner known per se. Microemulsions are firstly understood as meaning all macroscopically homogeneous, optically transparent, low viscosity and in particular thermodynamically stable mixtures of two immiscible liquids and at least one nonionic or one ionic surfactant. The average particle sizes of the microemulsions are usually below 100 nm, they have a high transparency and are stable against visible phase separation upon centrifugation at 2000 rpm for at least 30 minutes.

The preparation of the microemulsions in step 1 preferably takes place simply by mixing the oil phase with the other oil-soluble ingredients, heating the oil phase to above the melting point of all of the constituents and then adding the aqueous surfactant-containing phase. The thermodynamically stable microemulsion is then formed spontaneously, if appropriate it also being necessary to stir a little.

The microemulsion comprises a sugar surfactant, namely an alkyl (oligo)glycoside (also referred to below as “APG”), as obligatory constituent. Within the context of the present teaching, alkyl and/or alkenyl oligo-glucosides conform here to the formula R¹O-[G]ₚ in which R¹ is an alkyl and/or alkenyl radical having 4 to 22 carbon atoms, G is a sugar radical having 5 or 6 carbon atoms and p is numbers from 1 to 10. They can be obtained by the relevant methods of preparative organic chemistry. The alkyl and/or alkenyl oligoglycosides can be derived from aldoses or ketoses having 5 or 6 carbon atoms, preferably glucose. The preferred alkyl and/or alkenyl oligoglycosides are thus alkyl and/or alkenyl oligoglucosides. The index number p in the general formula (I) indicates the degree of oligomerization (DP), i.e. the distribution of mono- and oligoglycosides and is a number between 1 and 10. Whereas p in a given compound must always be an integer and here can in particular assume the values p=1 to 6, the value p for a specific alkyl oligoglycoside is an analytically determined calculable quantity which in most cases is a fraction. Preference is given to using alkyl and/or alkenyl oligoglycosides having an average degree of oligomerization p of from 1.1 to 3.0. From an applications point of view, preference is given to those alkyl and/or alkenyl oligoglycosides whose degree of oligomerization is less than 1.7 and is in particular between 1.2 and 1.4. APGs are present in the microemulsions according to the present invention in amounts between 10 and 20% by weight, in each case based on the total amount of the microemulsion. Particular preference is given here to amounts in the range from 14 to 19% by weight.

Furthermore, esters of fatty acids of chain length C12-C22 with glycerol are present in the emulsions according to the invention. Preference is given here to using monoesters of glycerol, with monoesters of glycerol with unsaturated linear fatty acids in particular being suitable. Within the context of the invention, particular preference is given to glycerol monooleate. These glycerol esters are present in the microemulsions in amounts of from 4 to 10% by weight, preferably 5 to 9% by weight—in each case based on the total weight of the microemulsion.

Finally, the microemulsions of the present invention also comprise an oil body, thus a non-water-soluble organic phase in amounts of from 5 to 30% by weight. Here, particularly preferred oil phase are selected from the group of Guerbet alcohols based on fatty alcohols having 6 to 18 carbon atoms, esters of linear C₆-C₂₂-fatty acids with linear or branched C₆-C₂₂-fatty alcohols or esters of branched C₆-C₁₃-carboxylic acids with linear or branched C₆-C₂₂-fatty alcohols, esters of linear C₆-C₂₂-fatty acids with branched alcohols, esters of C₆-C₂₂-fatty alcohols and/or Guerbet alcohols with aromatic carboxylic acids, triglycerides based on C₆-C₁₀-fatty acids, liquid mono-/di-/triglyceride mixtures based on C₆-C₁₈-fatty acids, esters of C₂-C₁₂-dicarboxylic acids with linear and branched alcohols having 1 to 22 carbon atoms or polyols having 2 to 10 carbon atoms and 2 to 6 hydroxyl groups, vegetable oils, branched primary alcohols, substituted cyclohexanes, linear and branched C₆-C₂₂-fatty alcohol carbonates, Guerbet carbonates based on fatty alcohols having 6 to 18, preferably 8 to 10, carbon atoms, esters of benzoic acid with linear and/or branched C₆-C₂₂-alcohols n linear or branched, symmetrical or asymmetrical dialkyl ethers having 6 to 22 carbon atoms per alkyl group, ring-opening products of epoxidized fatty acid esters with polyols, silicone oils and/or aliphatic or naphthenic hydrocarbons, dialkylcyclohexanes and/or silicone oils.

Hydrocarbons is the term used to refer to organic compounds which consist only of carbon and hydrogen. They include both cyclic and acyclic (=aliphatic) compounds. They comprise both saturated and also mono- or polyunsaturated compounds. The hydrocarbons may be linear or branched. Depending on the number of carbon atoms in the hydrocarbon, the hydrocarbons can be divided into odd-numbered hydrocarbons (such as, for example, nonane, undecane, tridecane) or even-numbered hydrocarbons (such as, for example, octane, dodecane, tetradecane). Depending on the type of branching, the hydrocarbons can be divided into linear (=unbranched) or branched hydrocarbons. Saturated, aliphatic hydrocarbons are also referred to as paraffins.

“Hydrocarbon mixture” is understood as meaning mixtures of hydrocarbons which comprise up to 10% by weight of substances which are not types of hydrocarbons. The % by weight data of the linear C11 and linear C11 hydrocarbons refers in each case to the sum of the hydrocarbons present in the mixture. The non-hydrocarbons present up to 10% by weight are not taken into consideration for this calculation.

The substances which are not types of hydrocarbons and which may be present in the hydrocarbon mixture up to 10% by weight, in particular up to 8% by weight, preferably up to 5% by weight, are, for example, fatty alcohols, which remain as unreacted starting materials in the hydrocarbon mixture.

The term “CX hydrocarbon” encompasses hydrocarbons with a carbon number of X, thus, for example, the term C11 hydrocarbon encompasses all hydrocarbons with a carbon number of 11.

Preference is given to hydrocarbon mixtures where the mixture comprises
(a) 50 to 90% by weight of linear C11 hydrocarbons, preferably n-undecane
(b) 10 to 50% by weight of linear C13 hydrocarbons, preferably n-tridecane
based on the sum of the hydrocarbons.

Furthermore, preference is given to a hydrocarbon mixture which comprises at least 2 different hydrocarbons whose carbon number differs by more than 1, where these 2 different hydrocarbons constitute at least 60% by weight, preferably at least 70% by weight—based on the sum of the hydrocarbons.

The term “2 different hydrocarbons” refers to hydrocarbons with a different carbon number.

This means if the hydrocarbon mixture comprises a hydrocarbon with a carbon number of n (n=integer), then the mixture also comprises at least one further hydrocarbon with a carbon number greater than or equal to n+2 or less than or equal to n−2.

Preferably, n is an odd number, in particular 7, 9, 11, 13, 15, 17, 19, 21 and/or 23.

Furthermore, the hydrocarbon used may be a hydrocarbon mixture which comprises ¹⁴C isotopes and where the hydrocarbon mixture comprises at least 2 different hydrocarbons whose carbon number differs by more than 1.

However, as oil component it is also possible to use solid fats and/or waxes. These may also be present in a mixture with the oils specified in the preceding section. Typical examples of fats are glycerides, i.e. solid or liquid vegetable or animal products which consist essentially of mixed glycerol esters of higher fatty acids. Here, mention is to be made in particular of solid mono- and diglycerides, such as, for example, glycerol monooleate or glycerol monostearate. Suitable waxes are, inter alia, natural waxes, such as, for example, candelilla wax, carnauba wax, Japan wax, esparto grass wax, cork wax, guaruma wax, rice germ oil wax, sugar cane wax, ouricury wax, montan wax, beeswax, shellac wax, spermaceti, lanolin (wool wax), uropygial wax, ceresine, ozokerite (earth wax), petrolatum, paraffin waxes, microwaxes; chemically modified waxes (hard waxes), such as, for example, montan ester waxes, sasol waxes, hydrogenated jojoba waxes, and also synthetic waxes, such as, for example, polyalkylene waxes and polyethylene glycol waxes. Tocopherols and essential oils are likewise suitable as oil component. The glycerol monoesters here, however, are not considered to be a constituent of the oil phase.

A further essential constituent of the microemulsions as are used in the method according to the invention is water. The water should preferably be demineralized. The microemulsions used in the first step of the method preferably comprise up to 81% by weight of water. Preferred ranges are amounts of from 30 to 80% by weight and in particular from 45 to 65% by weight of water.

Besides the ingredients described above, the microemulsions can also comprise, as additional constituent, fatty alcohols of the general formula R²—OH, where R² is a saturated or unsaturated, branched or unbranched alkyl or alkenyl radical having 6 to 22 carbon atoms, can comprise. Typical examples are caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol, and technical-grade mixtures thereof which are produced, for example, during the high-pressure hydrogenation of technical-grade methyl esters based on fats and oils or aldehydes from the Roelen oxo synthesis, and also as monomer fraction during the dimerization of unsaturated fatty alcohols. Preference is given to technical-grade fatty alcohols having 12 to 18 carbon atoms, such as, for example, coconut, palm, palm kernel or tallow fatty alcohol. Particular preference is given to the co-use of cetyl alcohol, stearyl alcohol, arachyl alcohol and behenyl alcohol, and mixtures thereof.

If fatty alcohols are present, they are preferably used in amounts up to 15% by weight, based on the total microemulsion, where the range from 1 to 10% by weight and preferably 2 to 8% by weight may be particularly preferred. According to the invention, these fatty alcohols, which constitute water-insoluble organic constituents, also do not fall under the definition of the oil body.

The microemulsion which is prepared in the first step of the method according to the invention can furthermore also comprise anionic surfactants. Typical examples of anionic surfactants are soaps, alkylbenzenesulfonates, alkanesulfonates, olefinsulfonates, α-methyl ester sulfonates, sulfo fatty acids, alkyl sulfates, alkyl ether sulfates, mono- and dialkyl sulfosuccinates, mono- and dialkyl sulfosuccinamates, sulfotriglycerides, monoglyceride sulfates, amide soaps, ether carboxylic acids and salts thereof, fatty acid isethionates, fatty acid sarcosinates, fatty acid taurides, N-acylamino acids, such as, for example, acyl lactylates, acyl tartrates, acyl glutamates and acyl aspartates, alkyl oligoglucoside sulfates and protein fatty acid condensates (in particular vegetable products based on wheat).

Within the context of the present invention, preference is given to fatty alcohol ether sulfates, here in particular to those of the general formula R³O—(CH₂CH₂O)ₘSO₃X, in which R³ is a linear or branched alkyl and/or alkenyl radical having 6 to 22 carbon atoms, n is numbers from 1 to 10 and X is an alkali metal and/or alkaline earth metal, ammonium, alkylammonium, alkanolammonium or glucammonium. Alkyl ether sulfates (“ether sulfates”) are known anionic surfactants which are prepared industrially by SO₃ or chlorosulfonic acid (CSA) sulfation of fatty alcohol or oxo alcohol polyglycol ethers and subsequent neutralization. Typical examples are the sulfates of addition products of, on average, 1 to 10 and in particular 2 to 5 mol of ethylene oxide onto caproic alcohol, capryl alcohol, 2-ethylhexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol, and also technical-grade mixtures thereof in the form of their sodium and/or magnesium salts. The ether sulfates here can have either a conventional or a narrowed homolog distribution.

Particular preference is given to the use of ether sulfates based on adducts of, on average, 2 to 3 mol of ethylene oxide onto technical-grade C_{12/14}- or C_{12/18}-coconut fatty alcohol fractions in the form of their sodium and/or magnesium salts.

The microemulsions used in the method according to the invention can also comprise further nonionic, amphoteric and/or cationic surfactants, preferably in amounts of, in total, 1 to 25% by weight, based on the total weight of the emulsion. Typical examples of further nonionic surfactants (besides the alkyl (oligo)glycosides) are, for example, fatty acid N-alkylglucamides, polyol fatty acid esters, sugar esters, sorbitan esters, polysorbates, alcohol ethoxylates and amine oxides. Depending on the preparation, alcohol ethoxylates are referred to as fatty alcohol ethoxylates or oxo alcohol ethoxylates and preferably conform to the formula R⁴O(CH₂CH₂O)ₙH R⁴ is a linear or branched alkyl and/or alkenyl radical having 6 to 22 carbon atoms and n is numbers from 1 to 50. Typical examples are the adducts of, on average, 1 to 50, preferably 5 to 40 and in particular 10 to 25, mol of caproic alcohol, capryl alcohol, 2-ethyl-hexyl alcohol, capric alcohol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol, and also technical-grade mixtures thereof which are produced, for example, during the high-pressure hydrogenation of technical-grade methyl esters based on fats and oils or aldehydes from the Roelen oxo synthesis, and also as monomer fraction in the dimerization of unsaturated fatty alcohols. Preference is given to adducts of from 10 to 40 mol of ethylene oxide onto technical-grade fatty alcohols having 12 to 18 carbon atoms, such as, for example, coconut, palm, palm kernel or tallow fatty alcohol.

Examples of suitable amphoteric and zwitterionic surfactants are alkylbetaines, alkylamidobetaines, aminopropionates, aminoglycinates, imidazolinium betaines and sulfobetaines. Examples of suitable alkyl-betaines are the carboxyalkylation products of secondary and in particular tertiary amines. Typical examples are the carboxymethylation products of hexylmethylamine, hexyldimethylamine, octyldimethylamine, decyldimethylamine, dodecylmethylamine, dodecyldimethylamine, dodecylethylmethylamine, C_{12/14}-cocoalkyldimethylamine, myristyldimethylamine, cetyldimethylamine, stearyldimethylamine, stearylethylmethylamine, oleyldimethylamine, C_{16/18}-tallow-alkyldimethylamine, and technical-grade mixtures thereof. Also suitable are furthermore carboxyalkylation products of amidoamines. Typical examples are reaction products of fatty acids having 6 to 22 carbon atoms, namely caproic acid, caprylic acid, capric acid, lauric acid, myristic acid, palmitic acid, palmoleic acid, stearic acid, isostearic acid, oleic acid, elaidic acid, petroselic acid, linoleic acid, linolenic acid, elaeostearic acid, arachic acid, gadoleic acid, behenic acid and erucic acid, and also technical-grade mixtures thereof, with N,N-dimethylaminoethylamine, N,N-dimethylaminopropylamine, N,N-diethylaminoethylamine and N,N-diethylaminopropylamine, which are condensed with sodium chloroacetate. Preference is given to using a condensation product of C_{8/18}-coconut fatty acid N,N-dimethylaminopropylamide with sodium chloroacetate. Furthermore, imidazolinium betaines are also suitable. These substances too are known substances which can be obtained, for example, by cyclizing condensation of 1 or 2 mol of fatty acid with polyfunctional amines, such as, for example, aminoethylethanolamine (AEEA) or diethylenetriamine. The corresponding carboxyalkylation products are mixtures of different open-chain betaines. Typical examples are condensation products of the aforementioned fatty acids with AEEA, preferably imidazolines based on lauric acid or again C_{12/14}-coconut fatty acid, which are then betainized with sodium chloroacetate.

Typical examples of cationic surfactants are quaternary ammonium compounds and ester quats, in particular quaternized fatty acid trialkanolamine ester salts.

Particularly preferred microemulsions for step 1 have the following composition:

| | |
|---|---|
| alkyl (oligo)glycosides | 10 to 20% by weight |
| glycerol fatty acid esters | 4 to 10% by weight |
| oil bodies | 5 to 30% by weight |
| fatty alcohol ether sulfates | 0 to 15% by weight |
| fatty alcohols | 0 to 15% by weight |

The remainder to 100% by weight is then in each case water, if appropriate supplemented by further, optional ingredients.

The microemulsions according to the above general description prepared in the first step of the method according to the invention are diluted in a separate step with water and then spontaneously form the finely divided emulsion according to the invention having an average particle size of less than 1 μm. Here—based on the volume of the microemulsion—5 to 20 times the volume of water is used for the dilution. The dilution step can be carried out at temperatures from 15 to 35° C., preferably at room temperature (=21° C.) Preferably, 4 to 9 parts of water are added per part of the microemulsion. The dilution step can take place directly after preparing the microemulsion in step 1—however, it is also possible and preferred in practice that the dilution takes place later. The microemulsions from step 1 are likewise storage-stable, meaning that, even upon prolonged storage of this intermediate, no disadvantages arise with regard to the stability or constitution of the emulsion prepared later in the second step.

The emulsion which are obtained by the final dilution step have an average particle size of less than 1 μm, preferably less than 0.8 μm. Preferably, the fraction of particles with a diameter or a size <1 μm is here at least 70%, preferably at least 80% and particularly preferably at least 90% of all particles. The finely divided emulsions prepared in this way are for their part provided by the present application.

The finely divided emulsions prepared according to the invention can be used for producing cosmetic preparations, such as, for example, hair shampoos, hair lotions, foam baths, shower gels, creams, gels, lotions, alcoholic and aqueous/alcoholic solutions, emulsions, wax/fatty masses, stick preparations, powders or ointments. These cosmetic compositions can also comprise, as further auxiliaries and additives, mild surfactants, oil bodies, emulsifiers, pearlescent waxes, consistency regulators, thickeners, superfatting agents, stabilizers, polymers, silicone compounds, fats, waxes, lecithins, phospholipids, biogenic active ingredients, UV sun protection factors, antioxidants, deodorants, antiperspirants, antidandruff agents, film formers, swelling agents, insect repellents, self-tanning agents, tyrosine inhibitors (depigmentation agents), hydrotropes, solubilizers, preservatives, perfume oils, dyes and the like. The application therefore further provides cosmetic compositions comprising an aqueous emulsion according to the above description. Preference is given to low viscosity lotions for the treatment of skin or hair.

### EXAMPLES

Firstly a microemulsion with the following composition was prepared by mixing the ingredients:

| | | % by weight of |
|---|---|---|
| Substance | INCI | active substance |
| Plantacare ® 2000 UP | Decyl Glucoside | 17.5 |
| Monomuls ® 90 O 18 | Glyceryl Oleate | 8 |
| Cetiol ® OE | Dicaprylyl Ether | 20 |
| Lanette ® O | Cetearyl Alcohol | 5 |
| Aqua dem. | | add 100 |

This microemulsion was then diluted with water in accordance with the invention. Here, three different dilutions were analyzed. The results are listed in the table below:

| Dilution with | | Average | Number of |
|---|---|---|---|
| demineralized | | particle size | particles |
| water | Appearance | [μm] | <1 μm |
| 80% dem. water | White-bluish | 0.61 ± 0.28 | 80% |
| 20% microemulsion | | | |
| 85% dem. water | White-bluish | 0.45 ± 0.35 | 88% |
| 15% microemulsion | | | |
| 90% dem. water | White-bluish | 0.45 ± 0.36 | 90% |
| 10% microemulsion | | | |

The particle size of the emulsions was measured using a measuring device of the type Horiba LB500. The emulsions obtained are storage-stable, i.e. upon storage at 40° C. over 4 weeks, no visible oil deposition occurred.

What is claimed is:

1. A method for preparing aqueous emulsions having an average particle size of less than 1 μm, comprising the steps of:
(1) providing a microemulsion comprising at least:
(a) 10-20% by weight of at least one alkyl (oligo)glycoside of the general formula R¹O-[G]ₚ in which R¹ is an alkyl and/or alkenyl moiety having 4 to 22 carbon atoms, G is a sugar moiety having 5 or 6 carbon atoms and p is a number from 1 to 10,
(b) 4-10% by weight of at least one ester of glycerol with a C12-C22 fatty acid,
(c) 5-30% by weight of at least one oil body, and
(d) the remainder to 100% by weight, water and, optionally, further ingredients, and
(2) diluting said microemulsion with water, using 5 to 20 times the volume of the microemulsion, at a temperature in the range of from 15 to 35° C.,
wherein the average particle size is less than 1 μm.

2. The method of claim 1, wherein said microemulsion further comprises at least one anionic, nonionic, amphoteric or cationic surfactant.

3. The method of claim 1 wherein said microemulsion further comprises at least one fatty alcohol of formula R²—OH, where R² is a saturated or unsaturated, branched or unbranched alkyl or alkenyl group having 6 to 22 carbon atoms.

4. The method of claim 1 wherein said microemulsion further comprises at least one fatty alcohol ether sulfate of formula R³O—(CH₂CH₂O)ₘSO₃X, in which R³ is a linear or branched alkyl and/or alkenyl group having 6 to 22 carbon atoms, n is a number from 1 to 10, and X is an alkali metal and/or alkaline earth metal, ammonium, alkylammonium, alkanolammonium or glucammonium group.

5. The method of claim 1 wherein said microemulsion comprises:
(a) 10 to 20% by weight of alkyl (oligo)glycosides,
(b) 4 to 10% by weight of glycerol fatty acid esters,
(c) 5 to 30% by weight of oil bodies,
(d) 0 to 15% by weight of fatty alcohol ether sulfates,
(e) 0 to 15% by weight of fatty alcohols, and
(f) the remainder to 100% water and, optionally, further ingredients.

6. The method of claim 1 wherein said oil body is selected from the group consisting of Guerbet alcohols based on fatty alcohols having 6 to 18 carbon atoms; esters of linear C₆-C₂₂-fatty acids with linear or branched C₆-C₂₂-fatty alcohols; esters of branched C₆-C₁₃-carboxylic acids with linear or branched C₆-C₂₂-fatty alcohols; esters of linear C₆-C₂₂-fatty acids with branched alcohols; esters of C₆-C₂₂-fatty alcohols and/or Guerbet alcohols with aromatic carboxylic acids; triglycerides based on C₆-C₁₀-fatty acids; liquid mono-/di-/triglyceride mixtures based on C₆-C₁₈-fatty acids; esters of C₂-C₁₂-dicarboxylic acids with linear or branched alcohols having 1 to 22 carbon atoms, or polyols having 2 to 10 carbon atoms and 2 to 6 hydroxyl groups; vegetable oils; branched primary alcohols; substituted cyclohexanes; linear and branched C₆-C₂₂-fatty alcohol carbonates; Guerbet carbonates based on fatty alcohols having 6 to 18 carbon atoms; esters of benzoic acid with linear and/or branched C₆-C₂₂-alcohols; linear or branched, symmetrical or unsymmetrical dialkyl ethers having 6 to 22 carbon atoms per alkyl group; ring-opening products of epoxidized fatty acid esters with polyols; silicone oils; aliphatic or naphthenic hydrocarbons; and dialkylcyclohexanes.

7. The aqueous emulsion having an average particle size of less than 1 μm, prepared according to claim 1.

8. A cosmetic composition comprising the aqueous emulsion of claim 7.

9. The aqueous emulsion of claim 7 wherein the average particle size is less than 0.8 μm.

10. A cosmetic composition comprising the aqueous emulsion of claim 9.
